(19) United States
(12) Patent Application Publication
(10) Pub. No.: US 2021/0228566 A1
(43) Pub. Date: Jul. 29, 2021

(54) METHODS OF TREATING A NEUROLOGICAL OR PSYCHIATRIC DISORDER

(71) Applicants: Guriqbal S. Nandra, Chicago, IL (US); Irvin M. Weisman, Chicago, IL (US)

(72) Inventors: Guriqbal S. Nandra, Chicago, IL (US); Irvin M. Weisman, Chicago, IL (US)

(21) Appl. No.: 15/998,635

(22) Filed: Aug. 16, 2018

(51) Int. Cl. A61K 31/4745

(52) U.S. Cl. CPC A61K 31/4745

(57) ABSTRACT

Provided are methods of administering a vesicular monoamine transport 2 (VMAT2) inhibitor chosen from valbenazine, or a pharmaceutically acceptable salt and/or isotopic variant thereof, to a patient in need thereof.

Dysregulation of dopaminergic systems is integral to several central nervous system (CNS) disorders, including neurological and psychiatric diseases and disorders. These neurological and psychiatric diseases and disorders include hyperkinetic movement disorders, and conditions such as schizophrenia and mood disorders. The transporter protein vesicular monoamine transporter-2 (VMAT2) plays an important role in presynaptic dopamine release and regulates monoamine uptake from the cytoplasm to the synaptic vesicle for storage and release.

Despite the advances that have been made in this field, there remains a need for new therapeutic products useful to treatment of neurological and psychiatric diseases and disorders and other related diseases or conditions described herein. One such agent is valbenazine, which has the following chemical structure:

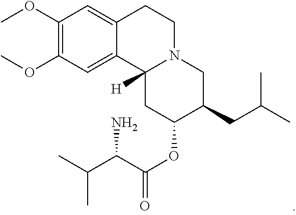 [SMILES: COc1cc2c(cc1OC)[C@H]1C[C@@H](OC(=O)[C@@H](N)C(C)C)[C@H](CC(C)C)CN1CC2]

A formulation of valbenazine:4-toluenesulfonate (1:2) (referred to herein as “valbenazine ditosylate”) has been previously reported in the FDA approved drug label Ingrezza®.

There is a significant, unmet need for methods for administering a VMAT2 inhibitor, such as valbenazine, or a pharmaceutically acceptable salt and/or isotopic variant thereof, to a patient in need thereof. The present disclosure fulfills these and other needs, as evident in reference to the following disclosure.

### BRIEF SUMMARY

Provided is a method of administering a vesicular monoamine transport 2 (VMAT2) inhibitor chosen from valbenazine, or a pharmaceutically acceptable salt and/or isotopic variant thereof, to a patient in need thereof, comprising:

administering a therapeutically effective amount of the VMAT2 inhibitor to the patient; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof who is at increased risk of one or more exposure-related adverse reactions, comprising:

administering a therapeutically effective amount of the VMAT2 inhibitor to the patient; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof who is at increased risk of one or more exposure-related adverse reactions, comprising:

administering an amount of the VMAT2 inhibitor to the patient that is reduced as compared to the amount that would be administered to a patient who is not at increased risk of one or more exposure-related adverse reactions; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof who has not experienced one or more exposure-related adverse reactions as a result of administration of the VMAT2 inhibitor wherein the one or more exposure-related adverse reactions is chosen from hypersensitivity and/or dermatological reactions, comprising:

administering a therapeutically effective amount of the VMAT2 inhibitor to the patient.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof; such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, “valbenazine” may be referred to as (S)-2-amino-3-methyl-butyric acid (2R, 3R,11bR)-3-isobutyl-9,10-dimethoxy-1,3,4,6,7,11b-hexahydro-2H-pyrido[2,1-a]isoquinolin-2-yl ester; or as L-Valine, (2R,3R,11bR)-1,3,4,6,7,11b-hexahydro-9,10-dimethoxy-3-(2-methylpropyl)-2H-benzo[a]quinolizin-2-yl ester or as NBI-98854.

As used herein, “(+)-α-HTBZ” means the compound which is an active metabolite of valbenazine having the structure:

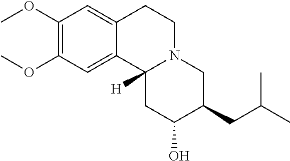 [SMILES: COc1cc2c(cc1OC)[C@H]1C[C@@H](O)[C@H](CC(C)C)CN1CC2]

(+)-α-HTBZ may be referred to as (2R, 3R, 11bR) or as (+)-α-DHTBZ or as (+)-α-HTBZ or as R,R,R-DHTBZ or as (+)-α-3-isobutyl-9,10-dimethoxy-1,3,4,6,7,11b-hexahydro-2H-pyrido[2,1-a]isoquinolin-2-ol; or as (2R, 3R,11bR)-3-isobutyl-9,10-dimethoxy-1,3,4,6,7,11b-hexahydro-2H-pyrido[2,1-a]isoquinolin-2-ol or as NBI-98782.

As used herein, “NBI-136110” means the compound which is a metabolite of valbenazine having the structure:

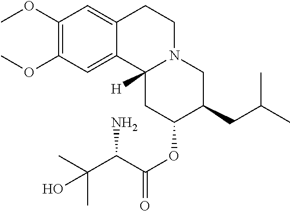 [SMILES: COc1cc2c(cc1OC)[C@H]1C[C@@H](OC(=O)[C@@H](N)C(C)(C)O)[C@H](CC(C)C)CN1CC2]

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a compound contains an unnatural proportion of deuterium.

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position. The isotopic enrichment of the compounds provided herein can be determined using conventional analytical methods known to one of ordinary skill in the art, including mass spectrometry, nuclear magnetic resonance spectroscopy, and crystallography.

As used herein, “hyperkinetic disorder” or “hyperkinetic movement disorder” or “hyperkinesias” refers to disorders or diseases characterized by excessive, abnormal, involuntary movements. These neurological disorders include tremor, dystonia, myoclonus, athetosis, Huntington's disease, tardive dyskinesia, Tourette syndrome, dystonia, hemiballismus, chorea, senile chorea, or tics.

As used herein, “tardive syndrome” encompasses but is not limited to tardive dyskinesia, tardive dystonia, tardive akathisia, tardive tics, myoclonus, tremor and withdrawal-emergent syndrome. Tardive dyskinesia is characterized by rapid, repetitive, stereotypic, involuntary movements of the face, limbs, or trunk.

As used herein, “about” means ±20% of the stated value, and includes more specifically values of ±10%, ±5%, ±2% and ±1% of the stated value.

As used herein, “AUC” refers to the area under the curve, or the integral, of the plasma concentration of an active pharmaceutical ingredient or metabolite over time following a dosing event.

As used herein “AUCo₀₋₄” is the integral under the plasma concentration curve from time 0 (dosing) to time “t”.

As used herein, “AUC_{0-∞}” is the AUC from time 0 (dosing) to time infinity. Unless otherwise stated, AUC refers to AUC_{0-∞}. Often a drug is packaged in a salt form, for example valbenazine ditosylate, and the dosage form strength refers to the mass of this salt form or the equivalent mass of the corresponding free base, valbenazine.

As used herein, Cₘₐₓ is a pharmacokinetic parameter denoting the maximum observed blood plasma concentration following delivery of an active pharmaceutical ingredient. Cₘₐₓ occurs at the time of maximum plasma concentration, tₘₐₓ.

As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

As used herein, “adjusting administration”, “altering administration”, “adjusting dosing”, or “altering dosing ” are all equivalent and mean tapering off, reducing or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein, 22q11.2 Deletion Syndrome (22q11.2 DS) is also known as Velocardiofacial syndrome (“VCFS”), DiGeorge syndrome, CATCH 22 and less often referred to as DiGeorge sequence, Microdeletion 22q11.2, Monosomy 22q11, Conotruncal anomaly face syndrome, Sedlac̆ková syndrome, Shprintzen syndrome, Takao syndrome, or Cayler cardiofacial syndrome. It is an autosomal dominant genetic condition that arises from the deletion of genes on chromosome 22 at band q11.2. Approximately 90% of individuals with VCFS have a 3 Mb deletion with the deletion of two genes, COMT and TBX1, being specifically associated with VCFS. Only ˜10% of individuals have a smaller 1.5 Mb deletion, which also typically includes the deletion of TBX1 and COMT. However, not all genes related to VCFS have been identified.

As used herein, “COMT” is a key enzyme for regulating catechol compounds, including dopamine, epinephrine and norepinephrine. Individuals with VCFS have approximately 50% less COMT mRNA, COMT protein expression, and enzyme activity compared to normal subjects. The characteristic behavioral manifestations of VCFS may be related to dopamine dysregulation resulting from COMT haploinsufficiency. However, that can be compounded by the presence of a low-activity COMT allele, leading to further dysregulation in patients with VCFS. COMT contains a common functional polymorphism, Va1158Met (r54680), which leads to alterations in enzyme activity. Individuals with VCFS who have a single copy of the Met allele have markedly low COMT activity. Compared with VCFS adults carrying the COMT Val allele, those carrying the Met allele tend to have increased risk for psychotic disorders, other neuropsychiatric syndromes, and have more severe cognitive deficits.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, a “dose” means the measured quantity of an active agent to be taken at one time by a patient. In certain embodiments, wherein the active agent is not valbenazine free base, the quantity is the molar equivalent to the corresponding amount of valbenazine free base. For example, often a drug is packaged in a pharmaceutically acceptable salt form, for example valbenazine ditosylate, and the dosage for strength refers to the mass of the molar equivalent of the corresponding free base, valbenazine. As an example, 73 mg of valbenazine tosylate is the molar equivalent of 40 mg of valbenazine free base.

As used herein, “dosing regimen” means the dose of an active agent taken at a first time by a patient and the interval (time or symptomatic) at which any subsequent doses of the active agent are taken by the patient such as from about 20 to about 160 mg once daily, e.g., about 20, about 40, about 60, about 80, about 100, about 120, or about 160 mg once daily. The additional doses of the active agent can be different from the dose taken at the first time.

As used herein, “effective amount” and “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein “hypersensitivity” or “hypersensitivity reaction” refers to an immunological sensitization due to a drug and/or its metabolites. Generally, there are four types of hypersensitivity:

Type I, IgE mediated—immediate-type hypersensitivity, including systemic hypersensitivity (e.g., anaphylaxis and urticarial) and respiratory hypersensitivity (e.g., asthma);

Type II, IgG or IgM mediated—antibody-mediated cytotoxic reaction and Type III, IgG mediated—immune complex reaction, which often occur simultaneously and are commonly associated with systemic or organ hypersensitivity reactions. Type II and III immunopathies include anemia, leukopenia, thrombocytopenia, pneumonitis, vasculitis, lupus-like reactions or glomeronephritis; and

Type IV, T lymphocyte mediated—delayed-type hypersensitivity response, which most commonly occurs as a delayed-type hypersensitivity skin reaction.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, a “medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means patient labeling for a pharmaceutical product approved by a regulatory agency (e.g., FDA, EMEA, or other equivalent regulatory agency) which contains information for patients on how to safely use a pharmaceutical product. A medication guide is scientifically accurate and is based on, and does not conflict with, the approved professional labeling for the pharmaceutical product, but the language need not be identical to the sections of approved labeling to which it corresponds. A medication guide is typically available for a pharmaceutical product with special risk management information.

As used herein, “ patient ” or “individual” or “ subject” means a mammal, including a human, for whom or which therapy is desired, and generally refers to the recipient of the therapy.

As used herein, “patient package insert” means information for patients on how to safely use a pharmaceutical product that is approved by a regulatory agency (e.g., FDA, EMEA, or other equivalent regulatory agency)). It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids or organic acids. Salts formed with the free carboxyl groups can also be derived from inorganic bases and organic bases. Lists of suitable salts are found in Remington's Pharmaceutical Sciences, 17.sup.th Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

Pharmaceutically acceptable salts can be formed from, for example, the following acids: 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); ascorbic acid (D); aspartic acid (L); aspartic acid (D); benzenesulfonic acid; benzoic acid; camphoric acid (+); camphoric acid (−); camphor-10-sulfonic acid (+); camphor-10-sulfonic acid (−); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); glucoheptonic acid (L); gluconic acid (D); gluconic acid (L); glucuronic acid (D); glucuronic acid (1); glutamic acid; glutaric acid; glycerophosphoric acid; glycolic acid; hippuric acid; hydrobromic acid; hydrochloric acid; isobutyric acid; lactic acid (DL); lactobionic acid; lauric acid; maleic acid; malic acid (−L); malic acid (D); malonic acid; mandelic acid (DL); methanesulfonic acid ; naphthalene-1,5-disulfonic acid; naphthalene-2-sulfonic acid; nicotinic acid; nitric acid; oleic acid; oxalic acid; palmitic acid; pamoic acid; phosphoric acid; proprionic acid; pyroglutamic acid (−L); pyroglutamic acid (D); salicylic acid; sebacic acid; stearic acid; succinic acid; sulfuric acid; tartaric acid (+L); tartaric acid (D); thiocyanic acid; toluenesulfonic acid (p); and/or undecylenic acid.

Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA, EMEA, or other equivalent regulatory agency) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “tₘₐₓ” is a pharmacokinetic parameter denoting the time to maximum blood plasma concentration following delivery of an active pharmaceutical ingredient

As used herein, “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

As used herein, “treating” or “treatment” refers to therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

As used herein, “VMAT2” refers to human vesicular monoamine transporter isoform 2, an integral membrane protein that acts to transport monoamines, particularly neurotransmitters such as dopamine, norepinephrine, serotonin, and histamine, from cellular cytosol into synaptic vesicles.

As used herein, the term “VMAT2 inhibitor”, “inhibit VMAT2”, or “inhibition of VMAT2” refers to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The term “VMAT2 inhibitor”, “inhibit VMAT2”, or “inhibition of VMAT2” also refers to altering the function of VMAT2 by decreasing the probability that a complex forms between a VMAT2 and a natural substrate.

Provided is a method of administering a vesicular monoamine transport 2 (VMAT2) inhibitor chosen from valbenazine, or a pharmaceutically acceptable salt and/or isotopic variant thereof, to a patient in need thereof, comprising:

administering a therapeutically effective amount of the VMAT2 inhibitor to the patient; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof who is at increased risk of one or more exposure-related adverse reactions, comprising:

administering a therapeutically effective amount of the VMAT2 inhibitor to the patient; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof who is at increased risk of one or more exposure-related adverse reactions, comprising:

administering an amount of the VMAT2 inhibitor to the patient that is reduced as compared to the amount that would be administered to a patient who is not at increased risk of one or more exposure-related adverse reactions; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof, comprising:

administering an initial dose of the VMAT2 inhibitor in the amount of 40 mg once daily and after one week, administering an increased dose of the VMAT2 inhibitor in amount of 80 mg once daily; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof who is at increased risk of one or more exposure-related adverse reactions, comprising:

administering an initial dose of the VMAT2 inhibitor in the amount of 40 mg once daily and after one week, administering an increased dose of the VMAT2 inhibitor in amount of 80 mg once daily; and

monitoring the patient for one or more exposure-related adverse reactions,

wherein the one or more exposure-related adverse reactions is chosen from hypersensitivity and/or dermatological reactions.

Also provided is a method of administering a vesicular monoamine transport 2 (VMAT2) inhibitor chosen from valbenazine, or a pharmaceutically acceptable salt and/or isotopic variant thereof, to a patient in need thereof who is at increased risk of one or more exposure-related adverse reactions, comprising:

administering an amount of the VMAT2 inhibitor to the patient that is reduced as compared to the amount that would be administered to a patient who is not at increased risk of one or more exposure-related adverse reactions, wherein the amount that would be administered to a patient who is not at increased risk comprises an initial dose of the VMAT2 inhibitor in the amount of 40 mg once daily and after one week, an increased dose of the VMAT2 inhibitor in amount of 80 mg once daily; and

monitoring the patient for one or more exposure-related adverse reactions,

wherein the one or more exposure-related adverse reactions is chosen from hypersensitivity and/or dermatological reactions.

In some embodiments, the method further comprises reducing the amount of the VMAT2 inhibitor based on the patient's ability to tolerate one or more exposure-related adverse reactions. In some embodiments, the amount of the VMAT2 inhibitor is reduced by at least about 10%. In some embodiments, the amount of the VMAT2 inhibitor is reduced by at least about 20%. In some embodiments, the amount of the VMAT2 inhibitor is reduced by at least about 30%. In some embodiments, the amount of the VMAT2 inhibitor reduced by at least about 40%. In some embodiments, the amount of the VMAT2 inhibitor is reduced by at least about 50%. In some embodiments, the amount of the VMAT2 inhibitor is reduced by at least about 60%. In some embodiments, the amount of the VMAT2 inhibitor is reduced by at least about 70%. In some embodiments, the amount of the VMAT2 inhibitor is reduced by at least about 80%. In some embodiments, the amount of the VMAT2 inhibitor is reduced by at least about 90%.

In certain embodiments, the therapeutically effective amount of the VMAT2 inhibitor is 10-90% less than the amount that would be administered to a patient who does not experience one or more exposure-related adverse reactions as a result of administration of the VMAT2 inhibitor.

In certain embodiments, the therapeutically effective amount of the VMAT2 inhibitor is 20-80% less than the amount that would be administered to a patient who does not experience one or more exposure-related adverse reactions as a result of administration of the VMAT2 inhibitor.

In certain embodiments, the therapeutically effective amount of the VMAT2 inhibitor is 30-70% less than the amount that would be administered to a patient who does not experience one or more exposure-related adverse reactions as a result of administration of the VMAT2 inhibitor.

In certain embodiments, the therapeutically effective amount of the VMAT2 inhibitor is 40-60% less than the amount that would be administered to a patient who does not experience one or more exposure-related adverse reactions as a result of administration of the VMAT2 inhibitor.

In certain embodiments, the therapeutically effective amount of the VMAT2 inhibitor is about 50% less than the amount that would be administered to a patient who does not experience one or more exposure-related adverse reactions as a result of administration of the VMAT2 inhibitor.

For example, where the amount that would be administered to a patient who does not experience one or more exposure-related adverse reactions as a result of administration of the VMAT2 inhibitor is 40 mg per day, an individual may receive a reduced dosage of 36, 32, 28, 24, 20, 16, 12, 8, or 4 mg per day. Likewise, where the amount that would be administered to a patient who does not experience one or more exposure-related adverse reactions as a result of administration of the VMAT2 inhibitor is 80 mg per day, an individual may receive a reduced dosage of 72, 64, 56, 48, 40, 32, 24, 16, or 8 per day.

For example, where the dosage administered to a patient who does not experience one or more exposure-related adverse reactions as a result of administration of the VMAT2 inhibitor is 40 mg per day, an individual may receive a reduced dosage of 4-36 mg per day, e.g., 8-32 mg per day, such as 12-28 mg per day, for example, 16-24 mg per day, or in certain embodiments, about 20 mg per day. Where the dosage administered to a patient who does not experience one or more exposure-related adverse reactions as a result of administration of the VMAT2 inhibitor is 80 mg per day, an individual may receive a reduced dosage of 8-72 mg per day, e.g., 16-64 mg per day, such as 24-56 mg per day, for example, 32-48 mg per day, or in certain embodiments, about 24 mg per day.

For example, where the dosage administered to a patient who does not experience one or more exposure-related adverse reactions as a result of administration of the VMAT2 inhibitor is 40 mg per day, an individual may receive a reduced dosage of 5-35 mg per day, e.g., 10-30 mg per day, such as 15-30 mg per day, for example, 15-25 mg per day, or in certain embodiments, about 20 mg per day or about 30 mg per day. where the dosage administered to a patient who does not experience one or more exposure-related adverse reactions as a result of administration of the VMAT2 inhibitor is 80 mg per day, an individual may receive a reduced dosage of 5-75 mg per day, e.g., 10-70 mg per day, such as 15-65 mg per day, for example, 20-60 mg per day, for example, 25-55 mg per day, for example, 30-50 mg per day, or in certain embodiments, about 40 mg per day.

In certain embodiments, the therapeutically effective amount is not reduced.

In some embodiments, the method further comprises discontinuing administration of the VMAT2 inhibitor based on the patient's ability to tolerate one or more exposure-related adverse reactions.

In some embodiments, the patient in need thereof who is at increased risk of one or more exposure-related adverse reactions has a history of allergies. In some embodiments, the patient has a history of allergies to one or more drugs, e.g., penicillin or paroxetine; to one or more types of food, e.g., eggs, milk, peanuts, tree nuts, fish, shellfish, wheat or soy; and/or to cats. In some embodiments, the patient has a history of hives.

In some embodiments, the method further comprises administering to the patient that is experiencing one or more exposure-related adverse reactions one or more medications chosen from steroids and antihistamines. In some embodiments, the steroid is a systemic glucocorticoid, such as prednisone. In some embodiments, the steroid is a hydrocortisone cream. In some embodiments, the antihistamine is diphenhydramine.

Also provided is a method of administering a vesicular monoamine transport 2 (VMAT2) inhibitor chosen from valbenazine, or a pharmaceutically acceptable salt and/or isotopic variant thereof, to a patient in need thereof who has not experienced one or more exposure-related adverse reactions as a result of administration of the VMAT2 inhibitor wherein the one or more exposure-related adverse reactions is chosen from hypersensitivity and/or dermatological reactions, comprising: administering a therapeutically effective amount of the VMAT2 inhibitor to the patient.

Also provided is a method of administering a vesicular monoamine transport 2 (VMAT2) inhibitor chosen from valbenazine, or a pharmaceutically acceptable salt and/or isotopic variant thereof, to a patient in need thereof who has not experienced one or more exposure-related adverse reactions as a result of administration of the VMAT2 inhibitor wherein the one or more exposure-related adverse reactions is chosen from hypersensitivity and/or dermatological reactions, comprising: administering an initial dose of the VMAT2 inhibitor in the amount of 40 mg once daily and after one week, administering an increased dose of the VMAT2 inhibitor in amount of 80 mg once daily.

In some embodiments, prior to the administration, the patient had not been administered the VMAT2 inhibitor.

In some embodiments, prior to the administration, the patient had been administered the VMAT2 inhibitor and patient had not experienced one or more exposure-related adverse events.

In some embodiments, prior to the administration, the patient had been administered the VMAT2 inhibitor and had experienced one or more exposure-related adverse events whereupon the amount of VMAT2 inhibitor administered to the patient was reduced and the patient subsequently did not experience one or more exposure-related adverse events at that reduced amount.

In some embodiments, the method further comprises informing the patient or a medical care worker that administration of the VMAT2 inhibitor to a patient may result in one or more exposure-related adverse reactions.

In some embodiments, the method further comprises informing the patient or a medical care worker that administration of the VMAT2 inhibitor to a patient may result in increased risk of one or more exposure-related adverse reactions.

In some embodiments, the method further comprises informing the patient to report to a medical care worker any exposure-related adverse reactions.

In some embodiments, the one or more exposure-related adverse reactions is chosen from hypersensitivity reactions. In some embodiments, the one or more exposure-related adverse reactions is chosen from hypersensitivity reactions with or without dermatological reactions. In some embodiments, the one or more exposure-related adverse reactions is chosen from hypersensitivity reactions with dermatological reactions. In some embodiments, the one or more exposure-related adverse reactions is chosen from hypersensitivity reactions without dermatological reactions. In some embodiments, the one or more exposure-related adverse reactions is chosen from allergic dermatitis, angioedema, pruritis, and urticaria.

In some embodiments, hypersensitivity is Type I hypersensitivity. In some embodiments, hypersensitivity is Type IV hypersensitivity.

In some embodiments, the one or more exposure-related adverse reactions is chosen from urticaria, pruritus, allergic dermatitis, and angioedema. In some embodiments, the one or more exposure-related adverse reactions is chosen from urticaria, allergic dermatitis, and angioedema. In some embodiments, the one or more exposure-related adverse reactions is hypersensitivity reaction and rash.

In some embodiments, the one or more exposure-related adverse reactions is chosen from rash, urticaria, and reactions consistent with angioedema.

In some embodiments, the one or more exposure-related adverse reactions is chosen from reactions consistent with angioedema. In some embodiments, the one or more exposure-related adverse reactions that are consistent with angioedema are chosen from swelling of the face, lips, and mouth, and dyspnea.

In some embodiments, the VMAT2 inhibitor is administered to the patient to treat a neurological or psychiatric disease or disorder. In some embodiments, the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis.

In some embodiments, the neurological or psychiatric disease or disorder is a hyperkinetic movement disorder. In some embodiments, the hyperkinetic movement disorder is tardive dyskinesia. In some embodiments, the hyperkinetic movement disorder is Tourette's syndrome. In some embodiments, the hyperkinetic movement disorder is Huntington's disease. In some embodiments, the hyperkinetic movement disorder is tics. In some embodiments, the hyperkinetic movement disorder is chorea associated with Huntington's disease. In some embodiments, the hyperkinetic movement disorder is ataxia, chorea, dystonia, Huntington's disease, myoclonus, restless leg syndrome, or tremors.

In some embodiments, the patient has been determined to have 22q11.2 deletion syndrome. In some embodiments, the patient is predisposed to developing a psychiatric disorder due to the patient having 22q11.2 deletion syndrome.

In some embodiments, the patient has been determined to have COMT haploinsufficiency. In some embodiments, the patient is predisposed to developing a psychiatric disorder due to the patient having COMT haploinsufficiency.

In some embodiments, the patient has a congenital defect of the cardiovascular system. In some embodiments, the patient has hypocalcemia and/or hypoparathyroidism. In some embodiments, the patient has hypothyroidism or hyperthyroidism. In some embodiments, the patient is obese. In some embodiments, the patient has recurrent seizures.

In some embodiments, the VMAT2 inhibitor is administered orally.

In some embodiments, the VMAT2 inhibitor is administered in the form of a tablet or capsule.

In some embodiments, the VMAT2 inhibitor is administered with or without food.

In some embodiments, the VMAT2 inhibitor is valbenazine or a pharmaceutically acceptable salt and/or isotopic variant thereof. In some embodiments, the VMAT2 inhibitor is valbenazine or a pharmaceutically acceptable salt thereof. In some embodiments, the VMAT2 inhibitor is a valbenazine tosylate salt. In some embodiments, the VMAT2 inhibitor is a ditosylate salt of valbenazine. In some embodiments, the VMAT2 inhibitor is an isotopic variant that is L-Valine, (2R,3R,11bR)-1,3,4,6,7,11b-hexahydro-9,10-di(methoxy-d3)-3-(2-methylpropyl)-2H-benzo[a]quinolizin-2-yl ester or a pharmaceutically acceptable salt thereof.

In some embodiments, the VMAT2 inhibitor is administered in an amount equivalent to between about 20 mg and about 160 mg of valbenazine free base. In some embodiments, the VMAT2 inhibitor is administered in an amount equivalent to about 20 mg of valbenazine free base. In some embodiments, the VMAT2 inhibitor is administered in an amount equivalent to about 40 mg of valbenazine free base. In some embodiments, the VMAT2 inhibitor is administered in an amount equivalent to about 60 mg of valbenazine free base. In some embodiments, the VMAT2 inhibitor is administered in an amount equivalent to about 80 mg of valbenazine free base. In some embodiments, the VMAT2 inhibitor is administered in an amount equivalent to about 120 mg of valbenazine free base.

In some embodiments, the VMAT2 inhibitor is administered for a first period of time in a first amount and then the amount is increased to a second amount. In some embodiments, the first period of time is a week. In some embodiments, the first amount is equivalent to about 40 mg of valbenazine free base. In some embodiments, the second amount is equivalent to about 80 mg of valbenazine free base.

In some embodiments, the VMAT2 inhibitor is administered in an amount sufficient to achieve a maximal blood plasma concentration (Cₘₐₓ) of (+)-α-DHTBZ of between about 15 ng to about 60 ng per mL plasma and a minimal blood plasma concentration (Cₘᵢₙ) of (+)-α-DHTBZ of at least 15 ng per mL plasma over an 8 hour period. In some embodiments, the VMAT2 inhibitor is administered in an amount sufficient to achieve a maximal blood plasma concentration (Cₘₐₓ) of (+)-α-DHTBZ of between about 15 ng to about 60 ng per mL plasma and a minimal blood plasma concentration (Cₘᵢₙ) of approximately between about at least 33% -50% of the Cₘₐₓ over a 12 hour period. In some embodiments, the VMAT2 inhibitor is administered in an amount sufficient to achieve: (i) a therapeutic concentration range of about 15 ng to about 60 ng of (+)-α-DHTBZ per mL plasma; and (ii) a threshold concentration of at least 15 ng (+)-α-DHTBZ per mL plasma over a period of about 8 hours to about 24 hours.

In certain embodiments, the Cₘₐₓ of R,R,R-DHTBZ is about 15 ng/mL, about 20 ng/mL, about 25 ng/mL, about 30 ng/mL, about 35 ng/mL, about 40 ng/mL, about 45 ng/mL, about 50 ng/mL, about 55 ng/mL or about 60 ng/mL plasma. In certain embodiments, the Cₘᵢₙ of R,R,R-DHTBZ is at least 15 ng/mL, at least 20 ng/mL, at least 25 ng/mL, at least 30 ng/mL, or at least 35 ng/mL plasma, over a period of 8 hrs, 12 hrs, 16 hrs, 20 hrs, 24 hrs, 28 hrs, or 32 hrs. In certain embodiments, the Cₘᵢₙ of R,R,R-DHTBZ is between about 15 ng/mL to about 35 ng/mL.

In certain embodiments, the pharmaceutical composition is administered in an amount sufficient to provide a Cₘₐₓ of R,R,R-DHTBZ of about 15 ng/mL to about 60 ng/mL plasma and a Cₘᵢₙ of approximately at least 33% of the Cₘₐₓ over a 24 hour period. In certain embodiments, the pharmaceutical composition is administered in an amount sufficient to provide a Cₘₐₓ of R,R,R-DHTBZ of about 15 ng/mL to about 60 ng/mL plasma and a Cₘᵢₙ of approximately at least 50% of the Cₘₐₓ over a 24 hour period. In certain embodiments, the pharmaceutical composition is administered in an amount sufficient to provide a Cₘₐₓ of R,R,R-DHTBZ of about 15 ng/mL to about 60 ng/mL plasma and a Cₘᵢₙ of approximately between about at least 33% -50% of the Cₘₐₓ over a 24 hour period.

In certain embodiments, the pharmaceutical composition is administered in an amount sufficient to provide a Cₘₐₓ of R,R,R-DHTBZ of about 15 ng/mL to about 60 ng/mL plasma and a Cₘᵢₙ of approximately at least 33% of the Cₘₐₓ over a 12 hour period. In certain embodiments, the pharmaceutical composition is administered in an amount sufficient to provide a Cₘₐₓ of R,R,R-DHTBZ of about 15 ng/mL to about 60 ng/mL plasma and a Cₘᵢₙ of approximately at least 50% of the Cₘₐₓ over a 12 hour period. In certain embodiments, the pharmaceutical composition is administered in an amount sufficient to provide a Cₘₐₓ of R,R,R-DHTBZ of about 15 ng/mL to about 60 ng/mL plasma and a Cₘᵢₙ of approximately between about at least 33% -50% of the Cₘₐₓ over a 12 hour period.

In certain embodiments, the pharmaceutical composition is administered to a subject in an amount that provides a Cₘₐₓ of R,R,R-DHTBZ of about 15 ng/mL to about 60 ng/mL plasma and a Cₘᵢₙ of between about 5 ng/mL to about 30 ng/mL plasma over a 24 hour period. In certain embodiments, the pharmaceutical composition is administered to a subject in an amount that provides a Cₘₐₓ of R,R,R-DHTBZ of about 15 ng/mL to about 60 ng/mL plasma and a Cₘᵢₙ of between about 7.5 ng/mL to about 30 ng/mL plasma over a 24 hour period.

In certain embodiments, a method for treating neurological or psychiatric diseases or disorders is provided herein that comprises administering to a subject a pharmaceutical composition comprising the VMAT2 inhibitor, as an active pharmaceutical ingredient, in an amount sufficient to provide: (i) a therapeutic concentration range of about 15 ng to about 60 ng of R,R,R-DHTBZ per mL plasma; and (ii) a threshold concentration of at least 15 ng R,R,R-DHTBZ per mL plasma over a period of about 8 hours to about 24 hours.

In certain embodiments, the therapeutic concentration range is about 15 ng to about 35 ng, to about 40 ng, to about 45 ng, to about 50 ng, or to about 55 ng R,R,R-DHTBZ per mL plasma.

In certain embodiments, the threshold concentration of R,R,R-DHTBZ is about 15 ng/mL, about 20 ng/mL, about 25 ng/mL, about 30 ng/mL, about 35 ng/mL, about 40 ng/mL, about 45 ng/mL, about 50 ng/mL, about 55 ng/mL or about 60 ng/mL plasma, over a period of about 8 hrs, about 12 hrs, about 16 hrs, about 20 hrs, about 24 hrs, about 28 hrs, or about 32 hrs. In certain embodiments, the threshold concentration of R,R,R-DHTBZ is between about 15 ng/mL to about 35 ng/mL over a period of about 8 hours to about 24 hours.

Plasma concentrations may be measured by methods known in the art and generally by tandem mass spectroscopy.

Valbenazine can be prepared according to U.S. Pat. Nos. 8,039,627 and 8,357,697, the disclosure of each of which is incorporated herein by reference in its entirety. Tetrabenazine may be administered by a variety of methods including the formulations disclosed in PCT Publications WO 2010/018408, WO 2011/019956, and WO 2014/047167, the disclosure of each of which is incorporated herein by reference in its entirety. In certain embodiments, the valbenazine for use in the compositions and methods provided herein is in polymorphic Form I as disclosed in U.S. Ser. No. 15/338,214, the disclosure of which is incorporated herein by reference in its entirety.

Also provided herein is a pharmaceutical composition for use in treating neurological or psychiatric diseases or disorders, comprising the VMAT2 inhibitor as an active pharmaceutical ingredient, in combination with one or more pharmaceutically acceptable carriers or excipients.

The choice of excipient, to a large extent, depends on factors, such as the particular mode of administration, the effect of the excipient on the solubility and stability of the active ingredient, and the nature of the dosage form.

The pharmaceutical compositions provided herein may be provided in unit dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein may be administered alone, or in combination with one or more other compounds provided herein, one or more other active ingredients. The pharmaceutical compositions provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art). The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pregelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Vee gum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as com starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL®200 (W. R. Grace Co., Baltimore, Md.) and CAB-0-SIL® (Cabot Co. of Boston, Mass.); and mixtures thereof. The pharmaceutical compositions provided herein may contain about 0.1 to about 5% by weight of a lubricant. Suitable glidants include colloidal silicon dioxide, CAB-0-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation. The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

In some embodiments, the pharmaceutical composition is prepared according to the methods described in U.S. Patent Appin. No. 62/561,629, filed Sep. 21, 2017 and/or U.S. Patent Appin. No. 62/564,951, filed Sep. 28, 2017, each of which is incorporated herein by reference for all purposes. In some embodiments, the pharmaceutical composition comprises valbenazine or a pharmaceutically acceptable salt thereof, silicified microcrystalline cellulose, isomalt, hydroxypropyl methylcellulose, partially pregelatinized maize starch, and magnesium stearate. In some embodiments, a unit dosage form of a pharmaceutical composition is provided with valbenazine ditosylate having a w/w% of at least 35%. In some embodiments, a unit dosage form of a pharmaceutical composition is provided with valbenazine ditosylate having a w/w% of about 40%, silicified microcrystalline cellulose having a w/w% of about 25%, isomalt having a w/w% of about 20%, hydroxypropyl methylcellulose having a w/w% of about 5%, partially pregelatinized maize starch having a w/w% of about 7.5%, and magnesium stearate having a w/w% of about 2.5%.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

In some embodiments, a unit dosage form of a pharmaceutical composition is provided having a capsule of size 1 or smaller and at least 80 mg of valbenazine or a pharmaceutically acceptable salt thereof, as measured as the free base. In some embodiments, a unit dosage form of a pharmaceutical composition is provided having a capsule of size 2 or smaller and at least 20 mg of valbenazine or a pharmaceutically acceptable salt thereof, as measured as the free base. In some embodiments, the unit dosage form has at least 40 mg of valbenazine or a pharmaceutically acceptable salt thereof, as measured as the free base. In some embodiments, a unit dosage form of a pharmaceutical composition is provided having a capsule of size 0 or smaller and at least 120 mg of valbenazine or a pharmaceutically acceptable salt thereof, as measured as the free base.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems.

The pharmaceutical compositions provided herein may be provided as noneffervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide. Coloring and flavoring agents can be used in all of the above dosage forms. The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and Hz-receptor antagonists.

The pharmaceutical compositions provided herein may be formulated as immediate or modified release dosage forms, including delayed-, sustained, pulsed-, controlled, targeted-, and programmed-release forms.

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile- or pulsed-, controlled-, accelerated- and fast-, targeted-, programmed-release, and gastric retention dosage forms.

The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

The pharmaceutical compositions provided herein in a modified release dosage form may be fabricated using a matrix controlled release device known to those skilled in the art.

### Dosages

In some embodiments, the VMAT2 inhibitor is administered at a daily dose of about 5 mg to about 160 mg. In some embodiments, the VMAT2 inhibitor is administered at a daily dose of about 5 mg. In some embodiments, the VMAT2 inhibitor is administered at a daily dose of about 20 mg. In some embodiments, the VMAT2 inhibitor is administered at a daily dose of about 40 mg. In some embodiments, the VMAT2 inhibitor is administered at a daily dose of about 60 mg. In some embodiments, the VMAT2 inhibitor is administered at a daily dose of about 80 mg. In some embodiments, the VMAT2 inhibitor is administered at a daily dose of about 100 mg. In some embodiments, the VMAT2 inhibitor is administered at a daily dose of about 120 mg. In some embodiments, the VMAT2 inhibitor is administered at a daily dose of about 140 mg. In some embodiments, the VMAT2 inhibitor is administered at a daily dose of about 160 mg.

For oral administration, the pharmaceutical compositions can be provided in the form of tablets containing 1.0 to 1,000 mg of the active ingredient, particularly about 1, about 5, about 10, about 15, about 20, about 25, about 30, about 40, about 45, about 50, about 75, about 80, about 100, about 150, about 200, about 250, about 300, about 400, about 500, about 600, about 750, about 800, about 900, and about 1,000 mg of the active ingredient for the symptomatic adjustment of the dosage to the patient to be treated. In certain embodiments, the pharmaceutical compositions can be provided in the form of tablets containing about 100 mg of the active ingredient. In certain embodiments, the pharmaceutical compositions can be provided in the form of tablets containing about 80 mg of the active ingredient. In certain embodiments, the pharmaceutical compositions can be provided in the form of tablets containing about 75 mg of the active ingredient. In certain embodiments, the pharmaceutical compositions can be provided in the form of tablets containing about 50 mg of the active ingredient. In certain embodiments, the pharmaceutical compositions can be provided in the form of tablets containing about 40 mg of the active ingredient. In certain embodiments, the pharmaceutical compositions can be provided in the form of tablets containing about 25 mg of the active ingredient. The compositions may be administered on a regimen of 1 to 4 times per day, including once, twice, three times, and four times per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds provided herein may also be combined or used in combination with other agents useful in the treatment, prevention, or amelioration of one or more symptoms of the diseases or conditions for which the compounds provided herein are useful, including tic disorders and other conditions commonly treated with antipsychotic medication.

In certain embodiments, the compounds provided herein may also be combined or used in combination with a typical antipsychotic drug. In certain embodiments, the typical antipsychotic drug is fluphenazine, haloperidol, loxapine, molindone, perphenazine, pimozide, sulpiride, thioridazine, or trifluoperazine. In certain embodiments, the antipsychotic drug is an atypical antipsychotic drug. In certain embodiments, the atypical antipsychotic drug is aripiprazole, asenapine, clozapine, iloperidone, olanzapine, paliperidone, quetiapine, risperidone, or ziprasidone. In certain embodiments, the atypical antipsychotic drug is clozapine.

Such other agents, or drugs, may be administered, by a route and in an amount commonly used thereof, simultaneously or sequentially with the compounds provided herein. When compounds provided herein are used contemporaneously with one or more other drugs, a pharmaceutical composition containing such other drugs in addition to the compounds provided herein may be utilized, but is not required. Accordingly, the pharmaceutical compositions provided herein include those that also contain one or more other active ingredients or therapeutic agents, in addition to the compounds provided herein.

The weight ratio of the compounds provided herein to the second active ingredient may be varied, and will depend upon the effective dose of each ingredient. Generally, an effective dose of each will be used. Thus, for example, when the compounds provided herein are used in combination with the second drug, or a pharmaceutical composition containing such other drug, the weight ratio of the particulates to the second drug may range from about 1,000:1 to about 1:1,000, or about 200:1 to about 1:200.

Combinations of the particulates provided herein and other active ingredients will generally also be within the aforementioned range, but in each case, an effective dose of each active ingredient should be used.

Also provided is a vesicular monoamine transport 2 (VMAT2) inhibitor chosen from valbenazine, or a pharmaceutically acceptable salt and/or isotopic variant thereof, for use in a method of treating a neurological or psychiatric disease or disorder of a patient in need thereof, said method comprising:
administering a therapeutically effective amount of the VMAT2 inhibitor to the patient; and
monitoring the patient for one or more exposure-related adverse reactions,
wherein the one or more exposure-related adverse reactions is chosen from hypersensitivity and/or dermatological reactions.

Also provided is a vesicular monoamine transport 2 (VMAT2) inhibitor chosen from valbenazine, or a pharmaceutically acceptable salt and/or isotopic variant thereof, for use in a method of treating a neurological or psychiatric disease or disorder of a patient in need thereof who is at increased risk of one or more exposure-related adverse reactions, said method comprising:
administering an amount of the VMAT2 inhibitor to the patient that is reduced as compared to the amount that would be administered to a patient who is not at increased risk of one or more exposure-related adverse reactions; and
monitoring the patient for one or more exposure-related adverse reactions,
wherein the one or more exposure-related adverse reactions is chosen from hypersensitivity and/or dermatological reactions.

Also provided is a vesicular monoamine transport 2 (VMAT2) inhibitor chosen from valbenazine, or a pharmaceutically acceptable salt and/or isotopic variant thereof, for use in a method of treating a neurological or psychiatric disease or disorder of a patient in need thereof who has not experienced one or more exposure-related adverse reactions as a result of administration of the VMAT2 inhibitor wherein the one or more exposure-related adverse reactions is chosen from hypersensitivity and/or dermatological reactions, said method comprising:

administering a therapeutically effective amount of the VMAT2 inhibitor to the patient.

In some embodiments, the method comprises selecting those patients experiencing one or more exposure-related adverse reactions chosen from hypersensitivity and/or dermatological reactions and reducing the amount of the VMAT2 inhibitor administered to the patient.

In some embodiments, the method comprises selecting those patients who are not experiencing one or more exposure-related adverse reactions is chosen from hypersensitivity and/or dermatological reactions for continued treatment with the VMAT2 inhibitor.

Also provided is a vesicular monoamine transport 2 (VMAT2) inhibitor chosen from valbenazine, or a pharmaceutically acceptable salt and/or isotopic variant thereof, for use in a method of

treating a neurological or psychiatric disease or disorder in a patient in need thereof, the method comprising administering to the individual an effective amount of the VMAT2 inhibitor, wherein said patient has been previously administered the VMAT2 inhibitor and monitored for one or more exposure-related adverse reactions chosen from hypersensitivity and/or dermatological reactions, and wherein said patient has been selected for treatment with the VMAT2 inhibitor in the absence of one or more exposure-related adverse reactions chosen from hypersensitivity and/or dermatological reactions.

Also provided is a method of selecting a patient for treatment with a vesicular monoamine transport 2 (VMAT2) inhibitor chosen from valbenazine, or a pharmaceutically acceptable salt and/or isotopic variant thereof, for treating a neurological or psychiatric disease or disorder comprising determining the presence or absence of one or more exposure-related adverse reactions chosen from hypersensitivity and/or dermatological reactions following administration of the VMAT2 inhibitor and selecting the patient being absent of one or more exposure-related adverse reactions chosen from hypersensitivity and/or dermatological reactions for treatment with the VMAT2 inhibitor.

Also provided is a vesicular monoamine transport 2 (VMAT2) inhibitor for use in any the methods disclosed herein.

Also provided is an article of manufacture comprising:
(i) a container
(ii) a composition within the container comprising a vesicular monoamine transport 2 (VMAT2) inhibitor chosen from valbenazine, or a pharmaceutically acceptable salt and/or isotopic variant thereof; and
(iii) a package insert instructing the user of the composition to administer the VMAT2 inhibitor to an individual suffering from a neurological or psychiatric disease or disorder, and warning the user of the composition of the risk of one or more exposure-related adverse reactions chosen from hypersensitivity and/or dermatological reactions.

Also provided is a kit for treating a neurological or psychiatric disease or disorder in an individual comprising a package having:
(i) a vesicular monoamine transport 2 (VMAT2) inhibitor chosen from valbenazine, or a pharmaceutically acceptable salt and/or isotopic variant thereof, and
(ii) instructions for using the VMAT2 inhibitor for treating a neurological or psychiatric disease or disorder, and warning the user of the composition of the risk of one or more exposure-related adverse reactions chosen from hypersensitivity and/or dermatological reactions.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1

### Pharmacologic Characterization of Valbenazine, Tetrabenazine, and Metabolite Thereof

Upon oral administration, TBZ is reduced to form four discrete isomeric secondary alcohol metabolites, collectively referred to as dihydrotetrabenazine (DHTBZ), which contains three asymmetric carbon centers (C-2, C-3, 0and C-11β), which could hypothetically result in eight stereoisomers. However, because the C-3 and C-11β carbons have fixed relative configurations, only four stereoisomers are possible: (R,R,R-DHTBZ or (+)-α-DHTBZ (alternate nomenclature) or NBI-98782 (laboratory nomenclature); S,S,S-DHTBZ or (−)-α-DHTBZ or NBI-98771; S,R,R-DHTBZ or (+)-β-DHTBZ or NBI-98795; and R,S,S-DHTBZ or (−)-β-DHTBZ or NBI-98772.

The affinity of each compound was measured by inhibition of [³H]-DHTBZ binding to rat forebrain membranes. The affinities relative to R,R,R-DHTBZ were also calculated and are presented. Data are reported as both the negative logarithm of the Ki (pKi) for statistical calculation with the normally distributed binding parameter used to determine the mean and SEM. The Ki value was determined from the mean pKi as 10(-pKi). The R,R,R-DHTBZ stereoisomer binds with the highest affinity to both rat and human VMAT2 (Ki =1.0 to 4.2 nM). In comparison, the remaining three DHTBZ stereoisomers (S,R,R-DHTBZ, S,S,S-DHTBZ, R,S,S-DHTBZ) bind to VMAT2 with a Ki values of 9.7, 250, and 690 nM, respectively.

### In Vitro VMAT2 Binding Affinity in Rat Forebrain

| | VMAT2 | | | |
|---|---|---|---|---|
| | | pKᵢ mean | | Affinity Relative to |
| Compound | Kᵢ,, nm | (SEM) | N | R,R,R-DHTBZ^{a} |
| R,R,R-DHTBZ | 4.2 | 8.38 (0.42) | 27 | 1.0 |
| S,R,R-DHTBZ | 9.7 | 8.01 (0.32) | 6 | 2.3 |
| S,S,S-DHTBZ | 250 | 6.60 (0.22) | 4 | 60 |
| R,S,S-DHTBZ | 690 | 6.16 (0.05) | 5 | 160 |

^{a}Affinity relative to R,R,R-DHTBZ was calculated using the Kᵢ value determined in the same study

The primary metabolic clearance pathways of valbenazine (VBZ, NBI-98854) are hydrolysis (to form R,R,R-DHTBZ) and mono-oxidation (to form the metabolite NBI-136110). R,R,R-DHTBZ and NBI-136110, the two most abundant circulating metabolites of VBZ, are formed gradually and their plasma concentrations decline with half-lives similar to VBZ.

VBZ and its metabolites, R,R,R-DHTBZ and NBI-136110, were tested for their ability to inhibit the binding of [3H]-DHTBZ to VMAT2 in cell lines or native tissues. The affinity of each compound was measured by inhibition of [³H]-DHTBZ binding to either human platelets or rat striatal membranes. The affinities relative to R,R,R-DHTBZ were also calculated and are presented. Data are reported as both the negative logarithm of the Kᵢ (pKi) for statistical calculation with the normally distributed binding parameter used to determine the mean and SEM (n=4 for each compound in each tissue). The Kᵢ value was determined from the mean pKi as 10^{(-pKi)}. The primary metabolite R,R,R-DHTBZ, was the most potent inhibitor of VMAT2 in rat striatum and human platelet homogenates.

### In Vitro VMAT2 Binding Affinity of Valbenazine and its Metabolites

| | Rat Striatum | | | Human Platelets | | |
|---|---|---|---|---|---|---|
| | | | Affinity | | | Affinity |
| | | pKᵢ mean | Relative to | | pKᵢ mean | Relative to |
| Compound | Kᵢ, nm | (SEM) | R,R,R-DHTBZ | Kᵢ, nm | (SEM) | R,R,R-DHTBZ |
| Valbenazine | 110 | 6.95 (0.02) | 39 | 150 | 6.82 (0.02) | 45 |
| R,R,R-DHTBZ | 1.98 | 8.70 (0.09) | 1.0 | 3.1 | 8.52 (0.03) | 1.0 |
| NBI-136610 | 160 | 6.80 (0.02) | 57 | 220 | 6.65 (0.04) | 67 |

VBZ and NBI-136110 had similar effects on VMAT2 inhibition, but with Ki values that were approximately 40-65 times the Ki values (lower affinity) of R,R,R-DHTBZ. These results were corroborated by the radioligand binding assay of DHTBZ stereoisomers (i.e., TBZ metabolites) in the rat forebrain, which also showed R,R,R-DHTBZ to be the most potent inhibitor of VMAT2, followed by S,R,R-DHTBZ. Comparatively, S,S,S-DHTBZ and R,S,S-DHTBZ, the other two primary metabolites of TBZ, were found to be poor VMAT2 inhibitors with affinities approximately 60 and 160 times weaker than R,R,R-DHTBZ.

The affinity of VBZ and its metabolites R,R,R-DHTBZ and NBI-136110 for other targets beyond VMAT2 was assessed in an extensive Cerep screen of multiple classes of protein targets including GPCRs, cell-surface monoamine transporters, and ion channels including the cardiac potassium channel, human ether-á-go-go-related gene (HERG).

The multi-target activity screen of more than 80 targets for these compounds (Cerep screen) demonstrated that VBZ and its metabolites, R,R,R-DHTBZ and NBI-136110, did not inhibit the binding of cognate ligands to any of the targets by more than 50% at concentrations of 1-10 μM. In contrast, the other three DHTBZ stereoisomers (S,R,R-DHTBZ, S,S,S-DHTBZ, R,S,S-DHTBZ), which are metabolites of TBZ but not VBZ, demonstrated >50% inhibition of ligand binding to a number of receptor subtypes including serotonin, dopamine and adrenergic receptors. Results expressed as percent of control specific binding: (tested compound specific binding/control specific binding)×100. All compounds were tested at 1 or 10 μM final concentration and results are an excerpt of a larger 80 target panel performed as an initial screen at Cerep (n=2 for each compound at each target). Bolded results (>50%) indicate activity at target receptor.

### In Vitro Activity of Valbenazine and DHTBZ Stereoisomers at Dopamine, Serotonin, and Adrenergic Receptors

| | | R,R,R- | S,R,R- | S,S,S-DHTBZ/ |
|---|---|---|---|---|
| Receptor Target | Valbenazine | DHTBZ | DHTBZ | R,S,S-DHTBZ^{a} |
| Serotonin5-HT_{1A} | 26 | 17 | 69 | 96 |
| Serotonin5-HT_{2A} | 1 | −4 | 3 | 84 |
| Serotonin5-HT₇ | 4 | 3 | 80 | 98 |
| Dopamine D₁ | 8 | −6 | −5 | 82 |
| Dopamine D₂₍ₛ₎ | 2 | 6 | 25 | 89 |

^{a}For purposes of the broad panel screen, the S,S,S- and R,S,S-metabolites were tested as a 50/50 mixture.

To describe the monoamine systems in greater detail, detailed radioligand binding assays were performed for dopamine, serotonin and adrenergic receptor subtypes as well as the transporters for dopamine (DAT), serotonin (SERT), and norepinephrine (NET) for the common metabolite of TBZ and VBZ (R,R,R-DHTBZ) and the other relevant metabolites unique to TBZ and VBZ. This detailed analysis revealed the high specificity of R,R,R-DHTBZ for the VMAT2 transporter and the non-specific activities of the other TBZ metabolites, including relatively high affinity for dopamine and serotonin receptor subtypes. Interestingly, the R,R,R-DHTBZ metabolite showed the greatest non-selectivity with respect to the monoamine receptors. None of the TBZ or VBZ metabolites had any affinity for the monoamine transporters DAT, SERT or NET. To complete the selectivity profile for VMAT2, the functional activity for the human VMAT1 transporter of these compounds was tested in cells expressing VMAT1. While the non-selective irreversible high-affinity uptake inhibitor of VMAT1, reserpine, substantially inhibited uptake through VMAT1, there was no significant inhibitory activity of TBZ, VBZ, or its metabolites R,R,R-DHTBZ or NBI-136110 at concentrations up to 10 μM. For both VMAT1 and VMAT2, uptake was measured in the untransfected host cells and was found to be similar to transfected cells in the presence of excess reserpine.

Radioligand binding assays and the broad panel screen indicate that in addition to varying potency at the VMAT2 transporter, two of the other DHTBZ metabolites of TBZ (S,S,S-DHTBZ and R,S,S-DHTBZ) interact with D1 and D2 receptors. Since VBZ is not metabolized to either of these DHTBZ stereoisomers, its effects on postsynaptic dopamine receptors either directly or indirectly through the metabolites are non-existent.

Moreover, results from the broad panel screen indicate that VBZ and its major metabolites (R,R,R-DHTBZ and NBI-136110) have little to no affinity for more than 80 binding sites, including receptors, monoamine transporters, and ion channels. This profile suggests a low potential for off-target pharmacological effects. In addition, uptake studies using TBZ, VBZ and its metabolites, R,R,R-DHTBZ and NBI-136110, confirmed the selectivity of these compounds for VMAT2 as they had no significant effect on the uptake of monoamines through VMAT1 compared to reserpine, a known VMAT1NMAT2 inhibitor.

The selectivity and specificity of VBZ was distinctively demonstrated using two in vivo surrogate measures of pharmacological effects. Ptosis, known to occur via adrenergic activation and prolactin release from the pituitary, modulated through the D2 dopamine receptor, demonstrated the difference between treatment with TBZ and VBZ. TBZ, VBZ and R,R,R-DHTBZ induced ptosis in an equivalent manner. This confirms that the metabolites formed by dosing TBZ or VBZ, or dosing of the active metabolite itself (R,R,R-DHTBZ) all have activity at VMAT2 affecting presynaptic monoamine release, in this case, related to norepinephrine release specifically to induce ptosis. Following similar treatment, but this time using prolactin release as a surrogate for dopaminergic modulation, R,R,R-DHTBZ and VBZ (to a lesser extent) induced a similar increase in serum prolactin levels as TBZ.

### Example 2

### Analysis of Hypersensitivity and Dermatological Reactions

A total of 17 cases of hypersensitivity reaction and/or dermatological findings and evidence for possible causal relationship with VBZ have been reported either spontaneously (6) or after solicitation (11). Of these 17 cases, 14 cases had both hypersensitivity reaction with dermatological findings (5 were serious and 9 were non-serious).

In these 14 cases, there was a reasonable temporal relationship between the initiation of the drug and the onset of events. In 1 case, both dechallenge and rechallenge was positive. In 2 cases, the events worsened when the drug was continued and the events resolved on discontinuation of VBZ. In 3 cases, dechallenge was considered as positive. In 7 cases, the event resolved or was resolving following discontinuation of drug and receiving appropriate treatment, implying a causal association. Additionally, among these 7 cases, there was 1 case in which the event re-occurred on readministration of the drug. In the remaining 1 case, the events started immediately after starting VBZ, the subject continued taking VBZ and the event continued; therefore, a causal association between the drug and the event could not be ruled out.

Of the 17 cases, there were 2 cases in which the patients experienced hypersensitivity reaction with evidence for a possible causal relationship with VBZ, but without dermatological findings. Both these cases were non-serious. In 1 case, dechallenge was positive and in another case, the event re-occurred with his second dose of drug, implying a causal association between the drug and the events.

Of the 17 cases, there was 1 case in which the patient experienced only dermatological findings with evidence for a possible causal relationship to VBZ, but without evidence of other hypersensitivity reaction. This case was non-serious and dechallenge was positive in this case. In view of the information reported in these 17 cases there appears to be a possible causal relationship between the events and administration of VBZ. However, it is important to note that no cases of severe hypersensitivity or dermatological reactions to suggest Stevens Johnson Syndrome, Toxic Epidermal Necrolysis, or Drug Rash with Eosinophilia and Systemic Symptoms have been reported with VBZ through 10 Oct. 2017.

Based on the case reports received through 10 Oct. 2017, it appears that VBZ may be associated with hypersensitivity reactions with or without dermatological reactions, including but not limited to hypersensitivity, rash, urticaria, pruritus, allergic dermatitis, and angioedema.

### Example 3

### Preparation of Capsule Containing 80mg Valbenazine

Capsules containing 80 mg valbenazine (measured as the free base) may be prepared according to the procedure set forth below, and the makeup of exemplary tablets are listed below.

| | Quantity 80 mg capsule | | |
|---|---|---|---|
| Component | (mg/capsule) | % (w/w) | Function |
| Valbenazine ditosylate | 145.80 | 40.0 | Active |
| Silicified Microcrystalline | 91.25 | 25.0 | Diluent |
| Cellulose | | | |
| Isomalt | 73.00 | 20.0 | Diluent |
| Partially pregelatinized | 27.38 | 7.5 | Disintegrant |
| maize starch | | | |
| Hydroxypropyl Methylcellulose | 18.25 | 5.0 | Binder |
| Magnesium stearate | 9.12 | 2.5 | Lubricant |
| Total Capsule Fill Weight | 364.80 | 100.00 | — |
| Hard gelatin capsule - Size #1 | 1 | — | Shell |

Valbenazine ditosylate, silicified microcrystalline cellulose (USP), isomalt (USNF), partially pregelatinized maize starch (USNF), hydroxypropyl methylcellulose (USNF) and magnesium stearate (USNF) were weighed according to the amounts above.

### Wallpapering:

Isomalt was transferred through a screening mill equipped with an 813 μm or equivalent round-hole screen to a tote bin for blending. The screened isomalt component was then blended.

### Pre-Blending and Screening:

The following components were transferred into the tote bin through a screening mill equipped with a ˜813 μm or equivalent round-hole screen: Valbenazine ditosylate and Silicified microcrystalline cellulose (“SMCC”). The components were then blended.

### Delumping:

The blend was vacuum transferred through a buffer tank equipped with a ˜813 μm or equivalent round-hole screen.

### Pre-Blending #2:

The screened components were again blended.

### Intragranular Blending:

The following components were then transferred, into the tote bin through a screening mill equipped with a ˜813 μm or equivalent round-hole screen:

a. Partially pregelatinized maize starch

b. Hydroxypropyl methylcellulose

The components were then blended. Inadequate de-agglomeration and subsequent dispersion of valbenazine ditosylate in isomalt and SMCC diluent can potentially impact content and uniformity of dosage units

### Lubricant Blending:

Magnesium stearate was manually screened (˜1 mm sieve) (intragranular quantity adjusted as needed based on pre-lubricated blend yield—limit 98%) into the opened tote bin for blending. The components were then blended. The desired output for this step is improved flowability with increased bulk and tapped density and improved particle size distribution.

### Roller Compaction:

The blend was then gravity fed through a roller compactor with a mill screen of 0.8-1.0 mm. The blend characterisics are important factors to consider for how well the blend will handle during encapsulation. Improper processing parameters can result in poor granule flow and compressibility which impacts encapsulation. The high solubility of the API and excipients should not impact dissolution. All roller compaction blends showed improvement over the initial intragranular blend properties which supports better capsule weight uniformity.

### Final Lubricant Blending:

Magnesium stearate was manually screened (˜1 mm sieve) (quantity to be adjusted as needed based on pre-lubricated blend yield—limit 98%) into the opened tote bin for blending. The components were then blended. The desired output for this step is a uniform and free flowing lubricated final blend for encapsulation. Inadequate blending can impact content and uniformity of dosage units. Over blending with hydrophobic magnesium stearate can impact dissolution. Blending is performed in a controlled environment minimizing moisture exposure.

### Encapsulation:

The lubricated blend was transferred to an automatic encapsulation machine and encapsulated into a size 1 capsule. Improper encapsulation equipment setup can impact filled capsule shell appearance. Capsule fill weight can impact content and dose uniformity. Capsule fill plug compression could impact dissolution and fill weight/content uniformity.

Encapsulation is performed in a controlled environment minimizing moisture exposure.

Dedusting and metal detection of the encapsulated product was performed, and the product was weight-checked.

### Example 4

### Preparation of 80 mg Valbenazine Capsule with Prior Art Formulation

An 80 mg dose formulation strategy attempted to use the known 40 mg capsule direct encapsulation formulation. Efforts were made to fill a Size 0 capsule with twice the amount of the 40 mg powder blend to yield an 80 mg strength capsule, as shown below.

| | Quantity 80 mg capsule | | |
|---|---|---|---|
| Component | (mg/capsule) | % (w/w) | Function |
| Valbenazine ditosylate | 146.0 | 28.21 | Active |
| Mannitol | 320.0 | 61.82 | Diluent |
| Partially pregelatinized | 40.0 | 7.73 | Disintegrant |
| maize starch | | | |
| Fumed silica | 6.4 | 1.24 | Glidant |
| Magnesium stearate | 2.4 | 1.00 | Lubricant |
| Total Capsule Fill Weight | 517.6 | 100.00 | — |
| Hard gelatin capsule - Size #0 | 1 | — | Shell |

Valbenazine ditosylate, mannitol (USP), partially pregelatinized maize starch (USNF), fumed silica (USNF) and magnesium stearate (USNF) were weighed according to the amounts shown above. A portion of the mannitol (¼) was transferred through a screening mill equipped with a ˜0.8 mm or equivalent round-hole screen to a tote bin for blending. The screened mannitol component was then blended.

### Pre-Blending and Screening:

The following components were transferred into the tote bin through a screening mill equipped with a ˜0.8 mm or equivalent round-hole screen:

a. Valbenazine ditosylate

b. Fumed silica

c. Partially pregelatinized maize starch

d. Remaining mannitol (¾)—(the adjustment of mannitol weight to compensate DS assay is performed on this fraction)

The components were blended and then transferred into polyethylene (PE) bags. The pre-blend was transferred through a screening mill equipped with an ˜0.8 mm or equivalent round-hole screen to a tote bin for blending.

### Final Lubricant Blending:

Magnesium stearate (quantity to be adjusted as needed based on pre-lubricated blend yield—limit 98%) was manually screened (˜1 mm sieve) into the opened tote bin for blending. The components were then blended.

### Encapsulation:

Efforts to fill a Size 0 capsule were unsuccessful. It was not possible to compress enough powder into a compact that would fit into a Size 0 capsule shell.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of administering a vesicular monoamine transport 2 (VMAT2) inhibitor chosen from valbenazine, or a pharmaceutically acceptable salt and/or isotopic variant thereof, to a patient in need thereof, comprising:
administering a therapeutically effective amount of the VAMT2 inhibitor to the patient; and
monitoring the patient for one or more exposure-related adverse reactions,
wherein the one or more exposure-related adverse reactions is chosen from hypersensitivity and/or dermatological reactions.

2.-117. (canceled)
